(19) United States
(12) Patent Application Publication
(10) Pub. No.: US 2022/0273267 A1
(43) Pub. Date: Sep. 1, 2022

(54) ULTRASONIC IMAGING METHOD AND ULTRASONIC IMAGING SYSTEM

(71) Applicant: GE Precision Healthcare LLC, Wauwatosa, WI (US)

(72) Inventors: Jiajiu Yang, Wuxi (CN); Zhiqiang Jiang, Wuxi (CN); Yao Ding, Wuxi (CN); Zhenyu Liu, Wuxi (CN)

(21) Appl. No.: 17/666,288

(22) Filed: Feb. 7, 2022

(30) Foreign Application Priority Data
Feb. 26, 2021 (CN) 202110220730.2

(51) Int. Cl. A61B 8/00

(52) U.S. Cl. CPC A61B 8/463; A61B 8/466

(57) ABSTRACT

Various ultrasonic imaging methods and ultrasonic imaging systems are provided. According to an aspect, a method includes acquiring first volumetric data collected by a probe at a first angle; generating a real-time ultrasonic image on the basis of the first volumetric data and displaying the same on a display device; and generating at least one ultrasonic image preview and at least one probe movement guide corresponding to the at least one ultrasonic image preview and displaying the same on the display device, wherein the at least one ultrasonic image preview corresponds to at least one other angle of the probe, and the at least one probe movement guide provides a visual indication for guiding the probe to move from the first angle to the at least one other angle.

### TECHNICAL FIELD

The present invention relates to the field of medical imaging, and in particular, to an ultrasonic imaging method and an ultrasonic imaging system.

### BACKGROUND

Ultrasonic imaging is a widely used imaging means. Ultrasonic imaging can perform real-time imaging of organs and soft tissues in the human body. Ultrasonic imaging uses real-time and non-invasive high-frequency sound waves to produce two-dimensional (2D) images, three-dimensional (3D) images, and/or four-dimensional (4D) images (i.e., real-time/continuous 3D images).

When a user uses an ultrasonic imaging device to perform real-time scanning, an ultrasonic image initially obtained may not meet requirements. In this case, it is usually necessary to move the position of a probe on the surface of a tissue to be scanned (for example, the angle of the probe), so as to acquire ultrasonic data required by the user and perform high-quality imaging. However, it is often difficult for the user to predict in which direction or to which position the probe is to be controlled to move in order to obtain a satisfactory ultrasonic image.

### SUMMARY

The aforementioned deficiencies, disadvantages, and problems are solved herein, and these problems and solutions will be understood through reading and understanding of the following description.

Some embodiments of the present invention provide an ultrasonic imaging method. The ultrasonic imaging method comprises: acquiring first volumetric data collected by a probe at a first angle; generating a real-time ultrasonic image on the basis of the first volumetric data and displaying the same on a display device; and generating at least one ultrasonic image preview and at least one probe movement guide corresponding to the at least one ultrasonic image preview and displaying the same on the display device, wherein the at least one ultrasonic image preview corresponds to at least one other angle of the probe, and the at least one probe movement guide provides a visual indication for guiding the probe to move from the first angle to the at least one other angle.

Some embodiments of the present invention provide an ultrasonic imaging device. The ultrasonic imaging device comprises: a probe, configured to collect ultrasonic data; a processor, configured to perform: acquiring first volumetric data collected by the probe at a first angle, generating a real-time ultrasonic image on the basis of the first volumetric data and displaying the same on a display device, and generating at least one ultrasonic image preview and at least one probe movement guide corresponding to the at least one ultrasonic image preview and displaying the same on the display device, wherein the at least one ultrasonic image preview corresponds to at least one other angle of the probe, and the at least one probe movement guide provides a visual indication for guiding the probe to move from the first angle to the at least one other angle; and the display device, configured to receive a signal from the processor for display.

Some embodiments of the present invention provide a non-transitory computer-readable medium, storing a computer program having at least one code segment executable by a machine to cause the machine to perform the following steps: acquiring first volumetric data collected by a probe at a first angle; generating a real-time ultrasonic image on the basis of the first volumetric data and displaying the same on a display device; and generating at least one ultrasonic image preview and at least one probe movement guide corresponding to the at least one ultrasonic image preview and displaying the same on the display device, wherein the at least one ultrasonic image preview corresponds to at least one other angle of the probe, and the at least one probe movement guide provides a visual indication for guiding the probe to move from the first angle to the at least one other angle.

It should be understood that the brief description above is provided to introduce in a simplified form some concepts that will be further described in the Detailed Description. The brief description above is not meant to identify key or essential features of the claimed subject matter. The scope is defined uniquely by the claims that follow the detailed description. Furthermore, the claimed subject matter is not limited to implementations that solve any disadvantages noted above or in any section of the present disclosure.

### BRIEF DESCRIPTION OF THE DRAWINGS

The present invention will be better understood by reading the following description of non-limiting embodiments with reference to the accompanying drawings, where

FIG. 1 is a schematic diagram of an ultrasonic imaging system according to some embodiments of the present invention;

FIG. 2 is a flowchart of an ultrasonic imaging method according to some embodiments of the present invention;

FIG. 3 is an exemplary display of an ultrasonic image generated on the basis of volumetric ultrasonic data according to some embodiments of the present invention;

FIG. 4 is an exemplary display for ultrasonic image previews related to the ultrasonic image of FIG. 3 and probe movement guides corresponding to the ultrasonic image previews in some embodiments of the present invention;

FIG. 5 is an exemplary display for ultrasonic image previews related to the ultrasonic image of FIG. 3 and probe movement guides corresponding to the ultrasonic image previews in some other embodiments of the present invention; and

FIG. 6 is another exemplary display for ultrasonic image previews related to the ultrasonic image of FIG. 3 and probe movement guides corresponding to the ultrasonic image previews according to the present invention.

### DETAILED DESCRIPTION

Specific implementations of the present invention will be described in the following. It should be noted that during the specific description of the implementations, it is impossible to describe all features of the actual implementations in detail in present invention for the sake of brief description. It should be understood that in the actual implementation of any of the implementations, as in the process of any engineering project or design project, a variety of specific decisions are often made in order to achieve the developer's specific objectives and meet system-related or business-related restrictions, which will vary from one implementation to another. Moreover, it can also be understood that although the efforts made in such development process may be complex and lengthy, for those of ordinary skill in the art related to content disclosed in the present invention, some changes in design, manufacturing, production or the like based on the technical content disclosed in the present disclosure are only conventional technical means, and should not be construed as that the content of the present disclosure is insufficient.

Unless otherwise defined, the technical or scientific terms used in the claims and the description are as they are usually understood by those of ordinary skill in the art to which the present invention pertains. “First”, “second” and similar words used in the present invention and the claims do not denote any order, quantity or importance, but are merely intended to distinguish between different constituents. The term “one”, “a(n)”, or a similar term is not meant to be limiting, but rather denote the presence of at least one. The term “include”, “comprise”, or a similar term is intended to mean that an element or article that appears before “include” or “comprise” encompasses an element or article and equivalent elements that are listed after “include” or “comprise”, and does not exclude other elements or articles. The term “connect”, “connected”, or a similar term is not limited to a physical or mechanical connection, and is not limited to a direct or indirect connection.

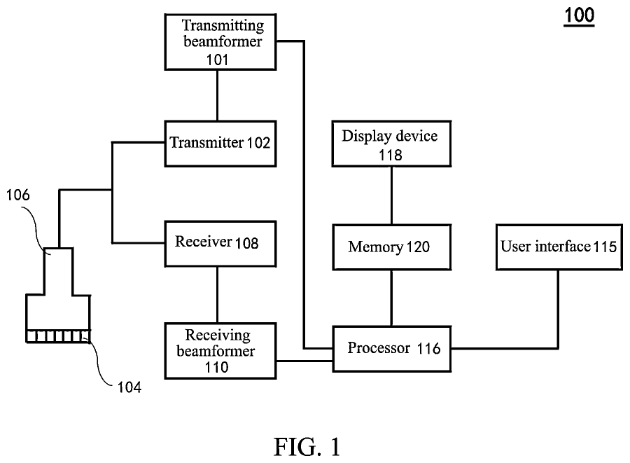

FIG. 1 is a schematic diagram of an ultrasonic imaging system 100 according to some embodiments of the present invention. The ultrasonic imaging system 100 includes a transmitting beamformer 101 and a transmitter 102, which drive elements 104 within a probe 106 to transmit ultrasonic pulse signals into the body (not shown). According to various embodiments, the probe 106 may be any type of probe including a linear probe, a curved array probe, a 1.25D array probe, a 1.5D array probe, a 1.75D array probe, or a 2D array probe. According to other embodiments, the probe 106 may also be a mechanical probe, for example, a mechanical 4D probe or a hybrid probe. The probe 106 may be configured to acquire 4D ultrasonic data, where the 4D ultrasonic data comprises information on how the volume changes over time. Each volume may include a plurality of 2D images or slices. Still referring to FIG. 1, the ultrasonic pulse signals are backscattered from structures in the body (for example, blood cells or muscle tissue) to produce echoes and return to the elements 104. The echoes are converted by the elements 104 into electrical signals or ultrasonic data, and the electrical signals are received by a receiver 108. The electrical signals representing the received echoes pass through a receiving beamformer 110 that outputs ultrasonic data. According to some embodiments, the probe 106 may include an electronic circuit to perform all or part of transmitting beamforming and/or receiving beamforming. For example, all or part of the transmitting beamformer 101, the transmitter 102, the receiver 108, and the receiving beamformer 110 may be located in the probe 106. The term “scan” or “scanning” may also be used in the present disclosure to refer to acquiring data through the process of transmitting and receiving ultrasonic signals. The terms “data” and “ultrasonic data” may be used in the present disclosure to refer to one or a plurality of datasets acquired using the ultrasonic imaging system. A user interface 115 may be configured to control operation of the ultrasonic imaging system 100. The user interface may be configured to control input of patient data, or select various modes, operations, parameters, and so on. The user interface 115 may include one or a plurality of user input devices, for example, a keyboard, hard keys, a touch pad, a touch screen, a trackball, a rotary control, a slider, soft keys, or any other user input device.

The ultrasonic imaging system 100 further includes a processor 116, which controls the transmitting beamformer 101, the transmitter 102, the receiver 108, and the receiving beamformer 110. According to various embodiments, the receiving beamformer 110 may be a conventional hardware beamformer or software beamformer. If the receiving beamformer 110 is a software beamformer, the receiving beamformer may include one or a plurality of the following components: a graphics processing unit (GPU), a microprocessor, a central processing unit (CPU), a digital signal processor (DSP), or any other type of processor capable of performing logical operations. The beamformer 110 may be configured to implement conventional beamforming techniques and techniques such as retrospective transmit beamformation (RTB).

The processor 116 is in electronic communication with the probe 106. The processor 116 may control the probe 106 to acquire ultrasonic data. The processor 116 controls which elements 104 are activated and the shape of a beam transmitted from the probe 106. The processor 116 is further in electronic communication with a display device 118, and the processor 116 may process the ultrasonic data into an image for display on the display device 118. For the purpose of the present disclosure, the term “electronic communication” may be defined to include wired connection and wireless connection. According to an embodiment, the processor 116 may include a central processing unit (CPU). According to other embodiments, the processor 116 may include other electronic components capable of performing processing functions, for example, a digital signal processor, a field-programmable gate array (FPGA), a graphics processing unit (GPU), or any other type of processor. According to other embodiments, the processor 116 may include a plurality of electronic components capable of performing processing functions. For example, the processor 116 may include two or more electronic components selected from a list including the following electronic components: a central processing unit (CPU), a digital signal processor (DSP), a field-programmable gate array (FPGA), and a graphics processing unit (GPU). According to another embodiment, the processor 116 may include a complex demodulator (not shown), which demodulates RF data and generates raw data. In another embodiment, the demodulation may be performed earlier in the processing chain. The processor 116 may be adapted to perform one or a plurality of processing operations on data according to a plurality of selectable ultrasound modalities. As echo signals are received, data may be processed in real time in a scanning stage. For the purpose of the present disclosure, the term “real time” is defined to include a process that is performed without any intentional delay. The real-time frame or volume rate may vary based on the site where data is acquired or the size of the volume and specific parameters used in the acquisition process. The data may be temporarily stored in a buffer (not shown) in the scanning stage, and processed in a less real-time manner in live or offline operations. Some embodiments of the present invention may include a plurality of processors (not shown) to cope with processing tasks. For example, a first processor may be configured to demodulate and decimate RF signals, while a second processor may be configured to further process data which is then displayed as an image. It should be recognized that other embodiments may use different processor arrangements. For embodiments where the receiving beamformer 110 is a software beamformer, the aforementioned processing tasks belonging to the processor 116 and the software beamformer herein may be performed by a single processor, for example, the receiving beamformer 110 or the processor 116. Alternatively, the processing functions belonging to the processor 116 and the software beamformer may be distributed among any number of separate processing components in a different manner.

According to an embodiment, the ultrasonic imaging system 100 may continuously acquire ultrasonic data at a frame rate of, for example, 10 Hz to 30 Hz. An image generated from the data may be refreshed at a similar frame rate. Data may be acquired and displayed at different rates in other embodiments. For example, depending on the size of the volume and potential applications, ultrasonic data may be acquired at a frame rate of less than 10 Hz or greater than 30 Hz in some embodiments. For example, many applications involve acquiring ultrasonic data at a frame rate of 50 Hz. A memory 120 is included therein to store processing frames for acquiring data. In an exemplary embodiment, the memory 120 has sufficient capacity to store ultrasonic data frames acquired over a period of time that are at least a few seconds long. The data frames are stored in a manner that facilitates retrieval according to the order or time of acquisition thereof. The memory 120 may include any known data storage medium.

Optionally, the embodiments of the present invention may be carried out using a contrast agent. When an ultrasound contrast agent including microbubbles is used, enhanced images of anatomical structures and blood flow in the body are generated by contrast imaging. After acquiring data using the contrast agent, image analysis includes: separating a harmonic component from a linear component, enhancing the harmonic component, and generating an ultrasonic image by using the enhanced harmonic component. Separation of the harmonic component from the received signal is performed using an appropriate filter. The use of a contrast agent in ultrasonic imaging is well known to those skilled in the art, and therefore is not described in further detail.

In various embodiments of the present invention, data may be processed by the processor 116 via modules of other or different related modes (for example, B-mode, color Doppler, M-mode, color M-mode, spectral Doppler, elastography, TVI, strain, strain rate, and so on) to form 2D or 3D images. For example, one or a plurality of modules may generate B-mode, color Doppler, M-mode, color M-mode, spectral Doppler, elastography, TVI, strain, strain rate, a combination thereof, and so on. Image bundles and/or frames are stored, and timing information indicating the time when data is acquired in the memory may be recorded. The module may include, for example, a scan conversion module that performs scan conversion operations to convert image frames from a coordinate bundle space to display space coordinates. A video processor module may be provided that reads image frames from the memory and displays the image frames in real time while performing operation on a patient. The video processor module may store image frames in an image memory, read images from the image memory, and display the images. The ultrasonic imaging system 100 may be a console-based system, a laptop computer, a handheld or portable system, or any other configuration.

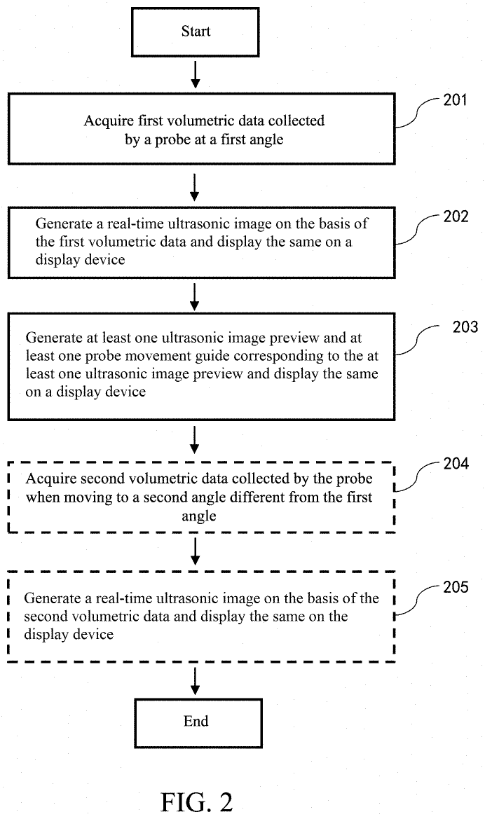

FIG. 2 is a flowchart of an ultrasonic imaging method 200 according to some embodiments of the present invention. Various modules in the flowchart represent steps that can be performed according to the method 200. Additional embodiments may perform the illustrated steps in a different order, and/or additional embodiments may include additional steps not shown in FIG. 2.

FIG. 2 is described in further detail below according to an exemplary embodiment. The method may be performed by the ultrasonic imaging system 100 shown in FIG. 1. For example, the method may be performed by the processor 116 in the ultrasonic imaging system 100.

In step 201, first volumetric data collected by a probe at a first angle is acquired. The obtaining process may be implemented by the aforementioned processor 116. For example, the processor 116 may obtain from the probe 106 ultrasonic data acquired from a body part of a person to be scanned. Generally, ultrasonic signals may be sent by the probe 106 to the tissue to be imaged, and then ultrasonic echo signals from the tissue to be imaged are received by the probe 106. The processor 116 can thereby acquire first volumetric ultrasonic data about the tissue to be imaged. The tissue to be imaged may be any human/animal tissue or organ. For example, the tissue to be imaged may be a liver, a kidney, a heart, a carotid artery, a breast, or the like.

A specific acquisition method of the volumetric ultrasonic data may be implemented by the method described above or by other methods. The aforementioned volumetric ultrasonic data may include 3D ultrasonic data or 4D ultrasonic data. The ultrasonic data is acquired and displayed in real time so as to serve as a part of the real-time ultrasonic imaging process.

The aforementioned first angle may be the inclination angle of the probe 106 relative to the surface of the tissue to be imaged, or the inclination angle of the probe relative to the organ to be scanned. In ultrasound scanning, there are usually specific requirements for a grip posture of a user holding the probe 106. The probe 106 usually has a mark to indicate whether the grip posture of the user is correct. That is, there is a default correlation between the directionality of the probe 106 and the obtained ultrasonic data and the orientation of an ultrasonic image generated from the data. Therefore, during the scanning, additional devices such as sensors are not required to detect the directions of forward, backward, leftward, and rightward movements of the probe, and there is a default correlation between an image display direction in the ultrasonic imaging system 100 and the direction and angle of the probe 106. In an alternative embodiment, sensors, such as an angular velocity sensor, a gyroscope, etc., may also be used to determine the angle and direction of movement of the probe 106.

In step 202, a real-time ultrasonic image is generated on the basis of the aforementioned first volumetric data and displayed on a display device. The process may be accomplished by the processor 116. Although the ultrasonic data acquired in the above step 201 is volumetric ultrasonic data, the real-time ultrasonic image generated using the volumetric ultrasonic data may be a 2D image, a 3D image, or a 4D image. For example, the image may be a B-mode image, a color Doppler image, an elastography image, a TVI image, or any other type of image generated from the ultrasonic data. According to an embodiment, the image may be a still frame generated from ultrasonic data. According to other embodiments, the processor 116 may generate images in two or more different imaging modes on the basis of the ultrasonic data in step 210. For example, in a VTI mode, the processor 116 may generate both a B-mode image and a spectral Doppler image based on the ultrasonic data. For example, in an IVC mode, the processor 116 may generate both a B-mode image and an M-mode image based on the ultrasonic data. In other embodiments, the ultrasonic image may include a volumetric ultrasonic image, such as a 3D image or a 4D image.

The ultrasonic image generated above is highly unlikely to meet needs of the user. For example, the angle of the image needs to be adjusted. Alternatively, a position of interest to be scanned is not or only part thereof is within the image range. In this case, a usual reaction of the user is to rotate or move the probe 106 to an appropriate position. However, the appropriate position is often difficult to determine, and the user needs to constantly make attempts and adjustments. The following steps of the present invention provide direct and convenient visual guidance for the user to adjust the probe in the real-time scanning process.

In step 203, at least one ultrasonic image preview and at least one probe movement guide corresponding to the at least one ultrasonic image preview are generated and displayed on the display device. The process may be accomplished by the processor 116. The aforementioned at least one ultrasonic image preview corresponds to at least one other angle of the probe 106. That is, the preview is an ultrasonic image that can be obtained by the processor 116 of the ultrasonic imaging system 100 through processing after the probe 106 moves to the at least one other angle and collects ultrasonic data. The generation of the ultrasonic image preview may be obtained by the processor 116 through calculation according to the volumetric data acquired in step 201 or the volumetric ultrasonic image generated in step 202. For example, the processor 116 may determine, according to the volumetric data, an ultrasonic image that can be obtained after the probe 116 is rotated forward, backward, leftward, or rightward (correspondingly, the ultrasonic image will also rotate). Alternatively, the processor 116 may make similar calculation and determination according to the generated volumetric ultrasonic image. Details will not be described herein again.

The aforementioned at least one probe movement guide provides a visual indication for guiding the probe to move from the first angle to the at least one other angle. The guidance may be implemented in various manners, which will be described in detail below. Such a configuration manner allows the user to intuitively observe which one or more of the aforementioned at least one ultrasonic image preview meets requirements of the user after seeing the at least one ultrasonic image preview, and in turn adjust a movement angle of the probe by means of the aforementioned at least one probe movement guide, to move the probe from the first angle to an angle corresponding to an ultrasonic image required by the user.

In some examples, the aforementioned at least one ultrasonic image preview and the at least one probe movement guide corresponding to the at least one ultrasonic image preview may each be singular. In other examples, the aforementioned at least one ultrasonic image preview and the at least one probe movement guide corresponding to the at least one ultrasonic image preview may each be plural. In addition, the at least one ultrasonic image preview and the at least one probe movement guide corresponding thereto may clearly correspond to each other, so as to facilitate direct observation by the user.

The aforementioned ultrasonic image preview and probe movement guide may be turned on/off in various manners. In some examples, the ultrasonic image preview and the probe movement guide may be generated automatically, without the need for additional operations by the user. Alternatively, in some other examples, the ultrasonic image preview and the probe movement guide may be turned on/off in response to operations of the user. For example, this may be controlled by inputting instructions through the user interface 115 above. Alternatively, in some scenarios, a probe 106 with keys or other input functions may be used, to use the input function to turn on/off the ultrasonic image preview and probe movement guide. Alternatively, this may be operated in voice, gesture input, and other manners.

It can be understood that in the real-time ultrasonic imaging process, once the angle and position of the probe 106 relative to the tissue to be imaged change, ultrasonic signals sent and received by the probe will also change accordingly. Accordingly, the processor 116 will acquire new ultrasonic data.

In view of this, some other examples of the present invention may further include: step 204, acquiring second volumetric data collected by the probe when moving to a second angle different from the first angle; and step 205, generating a real-time ultrasonic image on the basis of the second volumetric data and displaying the same on the display device. Similar to step 201 and step 202, these two steps may also be implemented by the processor 116.

The probe may be moved to the second angle in various manners. For example, the second angle may be obtained by the user by adjusting the angle of the probe 106 under the guidance of the ultrasonic image preview and the probe movement guide corresponding thereto given in step 203. Alternatively, the second angle may be obtained by adjusting the angle of the probe in any other manner by the user on his/her own. In some other examples, the second angle may also be automatically adjusted via a transmission device under control of the processor 116.

After the second volumetric data corresponding to the second angle of the probe is acquired, the processor 116 may generate a real-time ultrasonic image on the basis of the data and send an instruction to display the real-time ultrasonic image on the display device 118. Similar to the above steps, the real-time ultrasonic image may be a 2D image, a 3D image, or a 4D image. Details will not be described herein again.

Further, some other examples of the present invention may further include step 206: generating an updated ultrasonic image preview and an updated probe movement guide and displaying the same on the display device. This process is also implemented by the processor 116. It can be understood that the updated ultrasonic image preview and the updated probe movement guide will be performed on the basis of a current angle of the probe 106, so as to provide the user with new guidance. Specific generation methods thereof may be similar to those in step 203, which will not be repeated herein.

The updated ultrasonic image preview and the updated probe movement guide can ensure providing continuous guidance for the user, enabling the user to directionally move the ultrasonic probe until one or a plurality of ultrasonic images meeting requirements are obtained. For example, in an actual operation process of the user, an ultrasonic image and a corresponding ultrasonic image preview and probe movement guide will be continuously acquired in real time during the process of moving the probe. It should be noted that the above steps 204-206 may be implemented in the same example as steps 201-203, but are not necessary in the operations of the users. That is, steps 204-206 do not affect the integrity of implementation of steps 201-203 by the user. In addition, the above steps may also be repeated, for example, steps 204-206 are repeated a plurality of times until the ultrasonic scan is completed.

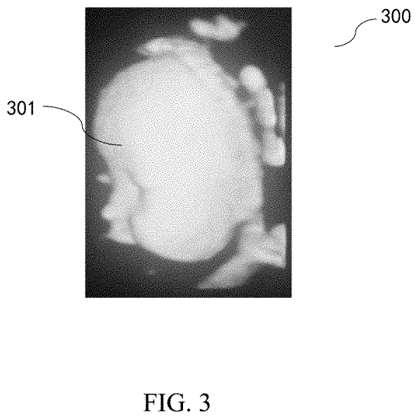

FIG. 3 shows an exemplary display of an ultrasonic image generated on the basis of volumetric ultrasonic data in some embodiments of the present invention. The ultrasonic image 300 includes a tissue 301 to be imaged (for example, the head of a fetus). The ultrasonic image 300 may correspond to the first angle of the probe 106, i.e., may be generated by processing the volumetric ultrasonic data received by the probe 106 at the first angle. The user usually needs some specific angles to better observe the tissue 301 to be imaged. In this case, the angle of the probe needs to be adjusted to adjust the real-time ultrasonic image.

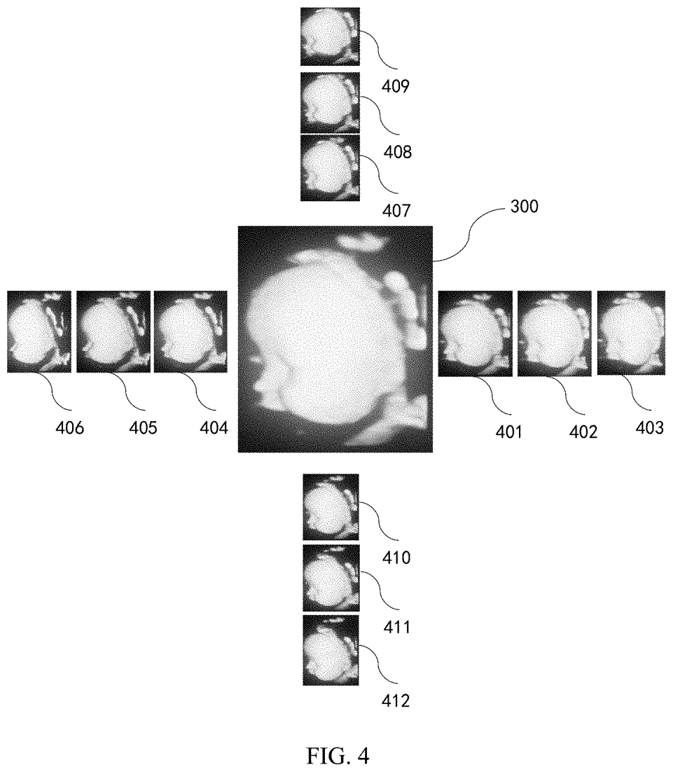

FIG. 4 shows an exemplary display of ultrasonic image previews related to the ultrasonic image of FIG. 3 and probe movement guides corresponding to the ultrasonic image previews in some embodiments of the present invention. As described above, a current angle of the tissue 301 to be imaged in the ultrasonic image 300 may be unfavorable for observation, and the user may need to adjust the angle of the probe. In this case, the user may choose to turn on an ultrasonic image preview and a probe movement guidance function corresponding thereto, or the function may be turned on automatically. As shown in FIG. 4, at least one ultrasonic image preview may be plural (for example, 12 as shown in FIG. 4), which are ultrasonic image previews 401-412, respectively. These ultrasonic image previews may be obtained by the processor through calculation on acquired volumetric ultrasonic data or generated volumetric ultrasonic images. The user can observe whether there is an image that meets scanning requirements thereof among these ultrasonic image previews. When a preview that meets the requirements is found, the user can move the probe according to a probe movement guide corresponding to the ultrasonic image preview, so as to adjust the real-time ultrasonic image to acquire an image satisfactory to the user.

The following is a detailed description of an example of the probe movement guide. As described above, the probe movement guide provides a visual indication for guiding the probe to move from the first angle to at least one other angle. This visual indication facilitates the user to intuitively understand how to move the probe. The visual indication may include a visual presentation of a relative position of an ultrasonic image preview to the ultrasonic image. Moreover, the visual presentation of the relative position of the ultrasonic image preview to the ultrasonic image corresponds to a direction of movement of the probe from the first angle to the at least one other angle.

The following is a further description with reference to FIG. 4. As shown in FIG. 4, the probe movement guide may include visual presentations of relative positions between the ultrasonic image previews 401-412 and the ultrasonic image 300. Specifically, the ultrasonic image previews 401, 402, and 403 are configured to be the right side of the ultrasonic image 300, and this relative position (right side) indicates that ultrasonic images represented by the ultrasonic image previews 401, 402, and 403 can be obtained by rotating the probe 106 to the right. Similarly, the ultrasonic image previews 404, 405, and 406 are configured to be the left side of the ultrasonic image 300, and this relative position (left side) indicates that ultrasonic images represented by the ultrasonic image previews 404, 405, and 406 can be obtained by rotating the probe 106 to the left. Similarly, the ultrasonic image previews 407-409 are configured to be the upper side the ultrasonic image 300, and this direction indicates an ultrasonic image that can be obtained by rotating the probe 106 upward. The ultrasonic image previews 410-412 are configured to be the lower side of the ultrasonic image 300, and this direction indicates an ultrasonic image that can be obtained by rotating the probe 106 downward.

It should be noted that the ultrasonic image previews 401-412 in FIG. 4 are respectively located in the upper, lower, left, and right directions of the ultrasonic image 300, but ultrasonic image previews in other directions may also be added, such as upper left, lower left, etc., which will not be repeated herein. In addition, although three ultrasonic image previews are generated in a certain rotation direction in FIG. 4, the number of previews may be arbitrary, for example, one, or any other number.

In addition, in the example shown in FIG. 4, the size of the ultrasonic image previews 401-412 is smaller than that of the ultrasonic image 300, thereby avoiding interference with observation of the ultrasonic image 300 by the user. In other examples, the size of the ultrasonic image previews may be equal to or greater than that of the ultrasonic image 300.

In some examples, the at least one other angle described above is set in advance. For example, there is at least one preset deflection angle fixed between the at least one other angle and the first angle which the probe is currently at. The processor 116 calculates a corresponding ultrasonic image preview according to the preset deflection angle. Correspondingly, the aforementioned at least one ultrasonic image preview is obtained after deflection by the preset at least one preset deflection angle according to the current ultrasonic image. It can be understood that when the aforementioned other angles include a plurality of angles, each of the plurality of other angles has a preset deflection angle with the aforementioned first angle. When a probe movement guide corresponding to an ultrasonic image preview is generated, a relative position of the ultrasonic image preview to the ultrasonic image is configured to correspond to the preset deflection angle direction, i.e., enabling a visual representation of a relative position of each of the at least one ultrasonic image preview to the ultrasonic image corresponds to a direction of movement of the probe from the first angle to the at least one other angle.

FIG. 4 is used as an example for detailed description. A plurality of deflection angles may be preset, for example, each including three angles of upward, downward, leftward, and rightward deflections (for example, 10°, 20°, and 30°), a total of 12 deflection angles, which corresponds to 12 other angles as described above. After the ultrasonic image 300 is generated, the processor automatically generates the ultrasonic image previews 401-412 corresponding to the 12 other angles of the ultrasonic image 300. In addition, these ultrasonic image previews 401-412 are respectively positioned relative to the ultrasonic image 300 in directions relative to the aforementioned 12 deflection angles. Visual presentations of relative positions of the ultrasonic image previews 401-412 to the ultrasonic image 300 corresponds to directions of movement of the probe from the current angle, i.e., the first angle, to the 12 other angles mentioned above.

In some examples, the probe movement guide may include only a visual presentation of the relative position between the ultrasonic image preview and the ultrasonic image as shown in FIG. 4. In other examples, in addition to the visual presentation of the relative position, the probe movement guide may further provide more detailed guidance. For example, the at least one probe movement guide may further include a visual presentation of a movement amount for guiding the probe to move from the aforementioned first angle to the aforementioned at least one other angle.

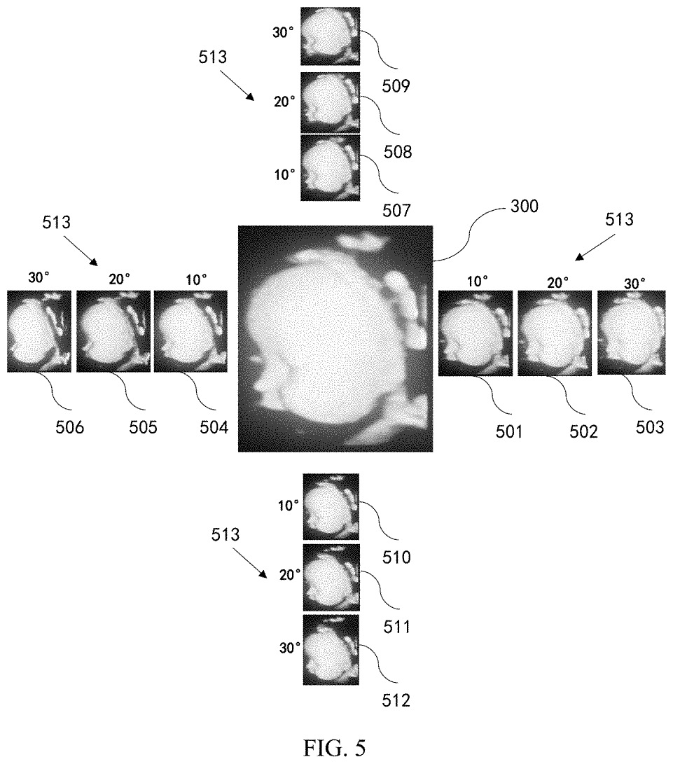

FIG. 5 is used for exemplary illustration. As shown in FIG. 5, there is shown an exemplary display of ultrasonic image previews related to the ultrasonic image of FIG. 3 and probe movement guides corresponding to the ultrasonic image previews in some other embodiments of the present invention. Similar to the example in FIG. 4, in this example, the probe movement guide may include visual presentations of relative positions between ultrasonic image previews 501-512 and the ultrasonic image 300. The difference lies in that, in the example of FIG. 5, the probe movement guide may also correspond to a visual presentation 513 of a movement amount of the probe corresponding to each of the ultrasonic image previews 501-512. Specifically, the visual presentation may be a specific probe rotation angle. For example, the ultrasonic image previews 501, 502, and 503 are ultrasonic images that can be obtained when the probe is deflected by 10°, 20°, and 30° to the right. Correspondingly, such movement amounts of “10°”, “20°”, and “30°” of the probe can be displayed on the display device, thereby serving as the visual presentations 513 of the corresponding movement amounts of the probe. This way of presentation can more intuitively prompt the user how to quantitatively move the probe.

It can be understood that the probe movement guide may not be limited to the forms described above herein, but may also include other forms, for example, visual representations using symbols such as arrows. Details will not be described herein again.

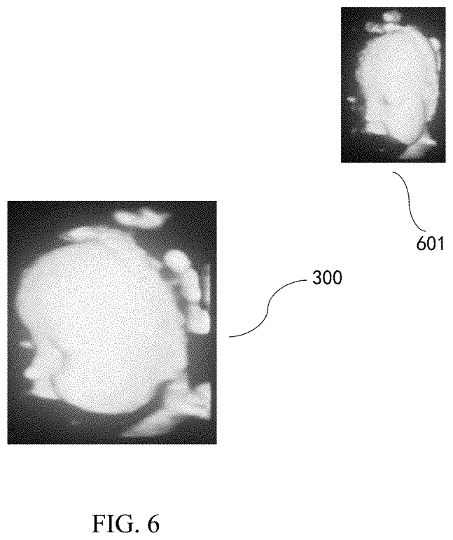

The above describes the generation of the ultrasonic image preview by using the preset deflection angle and the generation of the probe movement guide for indicating probe movement. In some other embodiments, these may also be implemented in other manner. Referring to FIG. 6, there is shown another exemplary display of ultrasonic image previews related to the ultrasonic image of FIG. 3 and probe movement guides corresponding to the ultrasonic image previews according to the present invention.

In the above other embodiments, generating at least one ultrasonic image preview may include: automatically identifying at least one anatomical region of interest in the ultrasonic image by using artificial intelligence, and then generating the at least one ultrasonic image preview of the at least one anatomical region of interest. The artificial intelligence may be implemented in various manners. In one example, the artificial intelligence may be implemented with a deep learning network by performing training using different volumetric ultrasonic image data sets as model input sets, and using anatomical regions of interest contained in these input images as model output sets. These anatomical regions of interest may be regions of great medical significance. For example, as shown in FIG. 6, the anatomical region of interest may be a frontal contour of a fetus. Alternatively, in other examples, the anatomical region of interest may be a specific perspective or region of other organs of clinical medical significance.

As discussed herein, the deep learning technology (also referred to as deep machine learning, hierarchical learning, deep structured learning, or the like) can employ a deep learning network (for example, an artificial neural network) to process input data and identify information of interest. The deep learning network may be implemented using one or a plurality of processing layers (such as an input layer, a normalization layer, a convolutional layer, a pooling layer, and an output layer, where processing layers of different numbers and functions may exist according to different deep learning network models), where the configuration and number of the layers allow the deep learning network to process complex information extraction and modeling tasks. Specific parameters (or referred to as “weight” or “bias”) of the network are usually estimated through a so-called learning process (or training process). The learned or trained parameters usually result in (or output) a network corresponding to layers of different levels, so that extraction or simulation of different aspects of initial data or the output of a previous layer usually may represent the hierarchical structure or concatenation of layers. During image processing or reconstruction, this may be represented as different layers with respect to different feature levels in the data. Thus, processing may be performed layer by layer. That is, “simple” features may be extracted from input data for an earlier or higher-level layer, and then these simple features are combined into a layer exhibiting features of higher complexity. In practice, each layer (or more specifically, each “neuron” in each layer) may process input data as output data for representation using one or a plurality of linear and/or non-linear transformations (so-called activation functions). The number of the plurality of “neurons” may be constant among the plurality of layers or may vary from layer to layer.

As part of initial training of the deep learning process to solve a specific problem, a training data set includes known input values (for example, known volumetric data or volumetric ultrasonic images) and desired (target) output values finally outputted from the deep learning process (for example, anatomical regions of interest contained in the known volumetric data or volumetric ultrasonic images). In this manner, a deep learning algorithm can process the training data set (in a supervised or guided manner or an unsupervised or unguided manner) until a mathematical relationship between a known input and an expected output is identified and/or a mathematical relationship between the input and output of each layer is identified and represented. In the learning process, (part of) input data is usually used, and a network output is created for the input data. Afterwards, the created network output is compared with the expected output of the data set, and then a difference between the created and expected outputs is used to iteratively update network parameters (weight and/or bias). A stochastic gradient descent (SGD) method may usually be used to update network parameters. However, those skilled in the art should understand that other methods known in the art may also be used to update network parameters. Similarly, a separate validation data set may be used to validate a trained learning network, where both a known input and an expected output are known. The known input is provided to the trained learning network so that a network output can be obtained, and then the network output is compared with the (known) expected output to validate prior training and/or prevent excessive training.

After identifying the aforementioned anatomical region of interest, the processor 116 will further use the volumetric data obtained by the probe 106 to generate at least one ultrasonic image preview 601 about the at least one anatomical region of interest.

Further, generating at least one probe movement guide corresponding to the aforementioned at least one ultrasonic image preview may include: determining at least one other angle of the probe corresponding to the at least one ultrasonic image preview of the at least one anatomical region of interest; and then generating a visual indication for guiding the probe to move from one angle to at least one other angle. As shown in FIG. 6, the visual indication may be shown, as described in the above embodiments, by positioning the at least one ultrasonic image preview 601 in a direction of movement from the first angle to the at least one other angle, so as to intuitively prompt the user. It should be noted that any configuration manner of the probe movement guide mentioned above can be applied to configuration of the ultrasonic image preview 601, which will not be repeated herein. Although FIG. 6 shows only one ultrasonic image preview 601, it can be understood that the number of previews may be arbitrary, for example, two or more.

By means of the artificial intelligence, the processor 116 can more directionally use the volumetric ultrasonic data or volumetric ultrasonic image to determine an ultrasonic image preview that is more valuable to the user, and can help the user adjust the position/angle of the ultrasonic probe more directionally.

Some embodiments of the present invention further provide an ultrasonic imaging system, which may be as shown in FIG. 1, or may be any other system. The system includes: a probe, configured to acquire ultrasonic data; and a processor, configured to perform the method in any of the embodiments described above. The system further includes a display device configured to receive a signal from the processor for display.

Some embodiments of the present invention further provide a non-transitory computer-readable medium storing a computer program, wherein the computer program has at least one code segment, and the at least one code segment is executable by a machine so that the machine performs steps of the method in any of the embodiments described above.

Accordingly, the present disclosure may be implemented in the form of hardware, software, or a combination of hardware and software. The present disclosure may be implemented in at least one computer system in a centralized manner, or in a distributed manner in which different elements are distributed on a number of interconnected computer systems. Any type of computer system or other device suitable for implementing the methods described herein are appropriate.

The various embodiments may also be embedded in a computer program product that includes all features capable of implementing the methods described herein, and the computer program product is capable of executing these methods when being loaded into a computer system. The computer program in this context means any expression in any language, code, or symbol of an instruction set intended to enable a system with information processing capabilities to execute a specific function directly or after any or both of a) conversion into another language, code, or symbol; and b) duplication in a different material form.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. An ultrasonic imaging method, comprising:
acquiring first volumetric data collected by a probe at a first angle;
generating a real-time ultrasonic image on the basis of the first volumetric data and displaying the real-time ultrasonic image on a display device; and
generating at least one ultrasonic image preview and at least one probe movement guide corresponding to the at least one ultrasonic image preview and displaying the at least one ultrasonic image preview and the at least one probe movement guide on the display device, wherein the at least one ultrasonic image preview corresponds to at least one other angle of the probe, and the at least one probe movement guide provides a visual indication for guiding the probe to move from the first angle to the at least one other angle.

2. The ultrasonic imaging method according to claim 1, wherein
the ultrasonic image comprises a volumetric ultrasonic image.

3. The ultrasonic imaging method according to claim 1, further comprising:
acquiring second volumetric data collected by the probe when moving to a second angle different from the first angle; and
generating a real-time ultrasonic image on the basis of the second volumetric data and displaying the same on the display device.

4. The ultrasonic imaging method according to claim 3, further comprising:
generating an updated ultrasonic image preview and an updated probe movement guide and displaying the same on the display device.

5. The ultrasonic imaging method according to claim 1, wherein
the at least one probe movement guide comprises a visual presentation of a relative position of the at least one ultrasonic image preview to the ultrasonic image, and the visual presentation of the relative position of the at least one ultrasonic image preview to the ultrasonic image corresponds to a direction of movement of the probe from the first angle to the at least one other angle.

6. The ultrasonic imaging method according to claim 5, wherein
the at least one probe movement guide further comprises a visual presentation of a movement amount for guiding the probe to move from the first angle to the at least one other angle.

7. The ultrasonic imaging method according to claim 1, wherein generating the at least one ultrasonic image preview comprises:
automatically identifying at least one anatomical region of interest in the ultrasonic image by using artificial intelligence; and
generating the at least one ultrasonic image preview of the at least one anatomical region of interest.

8. The ultrasonic imaging method according to claim 7, wherein generating the at least one probe movement guide corresponding to the at least one ultrasonic image preview comprises:
determining at least one other angle of the probe corresponding to the at least one ultrasonic image preview of the at least one anatomical region of interest; and
generating a visual indication for guiding the probe to move from the first angle to the at least one other angle.

9. The ultrasonic imaging method according to claim 1, wherein
at least one preset deflection angle exists between the at least one other angle and the first angle.

10. The ultrasonic imaging method according to claim 9, wherein generating the at least one probe movement guide corresponding to the at least one ultrasonic image preview comprises:
configuring a relative position of the at least one ultrasonic image preview to the ultrasonic image to correspond to the at least one preset deflection angle direction, such that a visual presentation of the relative position of the at least one ultrasonic image preview to the ultrasonic image corresponds to a direction of movement of the probe from the first angle to the at least one other angle.

11. (canceled)

12. (canceled)

13. An ultrasonic imaging system, comprising:
a probe;
a display device; and
a processor configured to:
control the probe to acquire first volume data at a first angle;
generate a real-time ultrasonic image based on the first volumetric data and display the real-time image on the display device; and
generate at least one ultrasonic image preview and at least one probe movement guide corresponding to the at least one ultrasonic image preview and display the ultrasonic image preview and the at least one probe movement guide on the display device, wherein the at least one ultrasonic image preview corresponds to at least one other angle of the probe, and the at least one probe movement guide provides a visual indication for guiding the probe to move from the first angle to the at least one other angle.

14. The ultrasonic imaging system of claim 13, wherein the ultrasonic image comprises a volumetric ultrasonic image.

15. The ultrasonic imaging system of claim 13, wherein the processor is further configured to:
control the probe to acquire second volumetric data when the probe is moved to a second angle different from the first angle;
generate an updated real-time ultrasonic image based on the second volumetric data and displaying it on the display device.

16. The ultrasonic imaging system of claim 15, wherein the processor is further configured to generate an updated ultrasonic image preview and an updated probe movement guide and display the updated ultrasonic image preview and the updated probe movement guide on the display device

17. The ultrasonic imaging system of claim 13, wherein the at least one probe movement guide comprises a visual presentation of a relative position of the at least one ultrasonic image preview to the ultrasonic image, and the visual presentation of the relative position of the at least one ultrasonic image preview to the ultrasonic image corresponds to a direction of movement of the probe from the first angle to the at least one other angle.

18. The ultrasonic imaging system of claim 17, wherein the at least one probe movement guide further comprises a visual presentation of a movement amount for guiding the probe to move from the first angle to the at least one other angle.

19. The ultrasonic imaging system of claim 13, wherein the processor is configured to generate the at least one ultrasonic image by:
automatically identifying at least one anatomical region of interest in the ultrasonic image by using artificial intelligence; and
generating the at least one ultrasonic image preview of the at least one anatomical region of interest.

20. The ultrasonic imaging system of claim 19, wherein the processor is configured to generate the at least one probe movement guide by:
determining at least one other angle of the probe corresponding to the at least one ultrasonic image preview of the at least one anatomical region of interest; and
generating a visual indication for guiding the probe to move from the first angle to the at least one other angle.

21. The ultrasonic imaging system of claim 13, wherein at least one preset deflection angle exists between the at least one other angle and the first angle.

22. The ultrasonic imaging system of claim 21, wherein the processor is configured to generate the at least one probe movement guide by configuring a relative position of the at least one ultrasonic image preview to the ultrasonic image to correspond to the at least one preset deflection angle direction, such that a visual presentation of the relative position of the at least one ultrasonic image preview to the ultrasonic image corresponds to a direction of movement of the probe from the first angle to the at least one other angle.
